(19) United States
(12) Patent Application Publication
(10) Pub. No.: US 2023/0002726 A1
(43) Pub. Date: Jan. 5, 2023

(54) METHODS OF IN-OVO SCREENING OF ANTI-CANCER THERAPIES

(71) Applicant: ICHILOV TECH LTD., Tel-Aviv (IL)

(72) Inventors: Nadir ARBER, Tel Aviv (IL); Shiran SHAPIRA, Petach-Tikva (IL); Oren BOGIN, Tel Aviv (IL)

(21) Appl. No.: 17/903,271

(22) Filed: Sep. 6, 2022

Related U.S. Application Data
(63) Continuation of application No. PCT/IL2021/050239, filed on Mar. 4, 2021.
(60) Provisional application No. 62/985,351, filed on Mar. 5, 2020.

(51) Int. Cl. C12N 5/073; C12N 5/00; G01N 33/50

(52) U.S. Cl. CPC C12N 5/0604; C12N 5/0068; G01N 33/5088

(57) ABSTRACT

Xenograft egg models comprising a fertilized non-mammalian egg comprising an ablated immune system, a first plurality of mammalian cells and a second plurality of mammalian cells, wherein the second plurality comprises immune cells are provided. Methods of producing the xenograft egg model as well as using the xenograft egg model for screening are also provided.

### CROSS REFERENCE TO RELATED APPLICATIONS

This application is a Bypass Continuation of PCT Patent Application No. PCT/IL2021/050239 having International filing date of Mar. 4, 2021, which claims the benefit of priority of U.S. Provisional Patent Application No. 62/985,351 titled METHODS OF IN-OVO SCREENING OF ANTI-CANCER THERAPIES, filed Mar. 5, 2020, the contents of which are all incorporated herein by reference in their entirety.

### FIELD OF INVENTION

The present invention is in the field of cancer diagnostics.

### BACKGROUND OF THE INVENTION

Predicting the clinical response to anti-cancer drugs remains a major challenge in cancer therapy. Everyday millions of people use medications that are not therapeutically beneficial or are even harmful. “Personalized medicine” is the tailoring of medical treatment to a single person, shaping the response to a particular disease and individual being treated. Accordingly, personalized medicine aims to increase target interference, while maximizing benefit and minimizing harmful events.

Several techniques are available for growing primary cell cultures from tumors. Immortalized cancer cell lines are readily available and provide an accessible, easily usable model of various cancer types which can be used to investigate cancer biology and the potential efficacy of anti-cancer therapeutics. In order to mimic the natural environment in which a cancer resides, several different methods have been implemented including the use of various culture matrices (like biomimetic scaffolds) and chemically defined media supplemented with essential nutrients for different tissues. However, these immortalized cell lines do not accurately represent the diversity, heterogeneity and drug-resistance of tumors typically occurring in patients. Furthermore, ex-vivo organ culture (EVOC) systems have been described which preserve the 3D structure, heterogeneity and complexity of the original tumor allowing tailoring of patient-specific therapies.

Animal models are the cornerstone of cancer research. Nude mice and patient-derived xenograft (PDX) models have been developed for research of various cancer types and for improvement of drug development. For example, PDX models are used for pre-clinical drug evaluation as they conserve the original tumor characteristics such as heterogeneity, complexity and molecular diversity. However, these methods are expensive, long in nature and agonizing to the model animals.

Recently, another in vivo model for cancer research has been proposed: the chick embryo chorioallantoic membrane (CAM) model. In this model, tumor cells are inoculated on the chorionic epithelium of the chick egg early after hatching (e.g., around embryo development day 3-5), for highly visible proliferation and invasion (see Xiao et al., PLoS One. (2015) 10(6): e0130935, herein incorporated by reference in its entirety). The chick embryo is initially naturally immunodeficient as its immune system reaches physiological activity only around day 15 post-fertilization. Thus, the CAM model can tolerate the transplantation of human tumor cells until the development of the chick immune system. The CAM model has been proposed in cancer research of various cancer types including breast, bladder, prostate, ovarian, and head and neck cancers for estimating the dissemination and angiogenesis of cancer cells (Xiao et al., PLoS One. (2015) 10(6): e0130935). The CAM model has also been proposed for generation of human Burkitt lymphoma xenograft tumors, which were compared with known characteristics of the human disease (Klingenberg et al., BMC Cancer (2014) 14:339). International Patent Publication WO/2017/079646 and U.S. Patent Application 2013/0171680 both also disclose the CAM model and its use. This model however has not been used to test immunotherapies as there is initially no immune system present in the model, and indeed the developing immune system is not useful as a stand in for the mature human immune system. In summary, the CAM model has been proposed for studying the growth, angiogenesis, invasion and metastasis of tumor cells. However, its uses have been up until now somewhat limited, and superior forms of the CAM model, specifically ones designed for the testing of immunotherapies are greatly needed.

### SUMMARY OF THE INVENTION

The present invention provides xenograft egg models comprising a fertilized non-mammalian egg comprising an ablated immune system, a first plurality of mammalian cells and a second plurality of mammalian cells, wherein the second plurality comprises immune cells are provided. Methods of producing the xenograft egg model as well as using the xenograft egg model for screening are also provided.

According to a first aspect, there is provided a xenograft egg model comprising:
a. a viable fertilized non-mammalian egg, wherein the egg comprises an ablated immune system;
b. a first plurality of mammalian cells in contact with a vasculature of the egg; and
c. a second plurality of mammalian cells in contact with a vasculature of the egg, wherein the second plurality of cells comprises immune cells from the same species as the mammalian cells of the first plurality.

According to some embodiments, the non-mammalian egg is an avian egg or a reptilian egg.

According to some embodiments, the avian egg is selected from the group consisting of a chicken egg, a turkey egg, a duck egg and a goose egg.

According to some embodiments, the first plurality of mammalian cells are in contact with a chorioallantoic membrane (CAM) of the egg, the second plurality of mammalian cells are in contact with a CAM of the egg or both.

According to some embodiments, the first plurality of mammalian cells, the second plurality of mammalian cells or both are located apically on the CAM.

According to some embodiments, the first plurality of mammalian cells and the second plurality of immune cells are separated by a distance of less than 1 cm.

According to some embodiments, the first plurality of mammalian cells comprises diseased cells.

According to some embodiments, the diseased cells are cancer cells.

According to some embodiments, the cancer is selected from the group consisting of colon cancer, colorectal cancer, lung cancer, prostate cancer, breast cancer, pancreatic cancer, liver cancer, kidney cancer, skin cancer, thyroid cancer, throat cancer, head or neck cancer, brain cancer, ovarian cancer, cervix cancer, spleen cancer, lymphoid cancer and hematopoietic cancer.

According to some embodiments, the mammal is a human.

According to some embodiments, the first plurality of mammalian cells, the second plurality of human cells, or both are comprised in a composition which comprises an exogenous matrix material.

According to some embodiments, the composition comprises hyaluronic acid (HA) or is Matrigel.

According to some embodiments, the composition comprises exogenous angiogenic growth factors.

According to some embodiments, the growth factors comprise bFGF and VEGF.

According to some embodiments, the immune cells comprise peripheral blood mononuclear cells (PBMCs).

According to some embodiments, the first plurality of mammalian cells and the second plurality of mammalian cells are derived from the same subject.

According to some embodiments, the egg comprising an ablated immune system is substantially devoid of CD45+ non-mammalian cells.

According to some embodiments, the egg is a chicken egg and remains devoid of CD45+ non-mammalian cells beyond day 15 of development.

According to some embodiments, the egg comprising an ablated immune system is an irradiated egg.

According to some embodiments, the mammalian cells of the second plurality of mammalian cells are capable of migrating to the first plurality of mammalian cells via the vasculature of the egg.

According to some embodiments, the first plurality of mammalian cells and the second plurality of mammalian cells are not in direct contact.

According to another aspect, there is provided a method of generating a xenograft egg model, the method comprising:
a. providing a viable fertilized non-mammalian egg;
b. irradiating the fertilized egg;
c. placing a first plurality of mammalian cells in contact with vasculature of the fertilized egg; and
d. placing a second plurality of mammalian cells in contact with vasculature of the fertilized egg wherein the second plurality of mammalian cells comprises immune cells from the same species as the mammalian cells of the first plurality,

thereby producing a xenograft egg model.

According to some embodiments, the egg comprises an eggshell and further comprising prior to (b) removing at least a part of egg albumen from a bottom region of the fertilized egg so as to separate a chorioallantoic membrane (CAM) of the fertilized egg from the eggshell; and creating an aperture at a top region of the eggshell to expose at least a portion of the CAM of the fertilized egg.

According to some embodiments, the removing at least part of the egg albumen, the creating an aperture at the top part of the eggshell, or both is performed on embryo development day 1-3.

According to some embodiments, the method of the invention further comprises resealing the aperture to segregate the fertilized egg inside the eggshell from an environment outside of the eggshell.

According to some embodiments, the first plurality of mammalian cells, the second plurality of mammalian cells or both are placed in contact with the CAM.

According to some embodiments, the irradiating is performed on embryo development day 4 to 7.

According to some embodiments, the irradiating is performed on embryo development day 5 or 6.

According to some embodiments, the irradiating comprises irradiating at 1.5-3.5 Gy at a rate of about 10-20 rad/sec.

According to some embodiments, the irradiating comprises irradiating at 2.5 Gy at a rate of about 15 rad/sec.

According to some embodiments, the method of the invention further comprises activating the CAM prior to placing the first plurality of mammalian cells, the second plurality of mammalian cells or both.

According to some embodiments, the method of the invention further comprises adding trypsin to the CAM prior to placing the first plurality of mammalian cells, the second plurality of mammalian cells or both.

According to some embodiments, the placing the first plurality of mammalian cells is performed on embryo development day 6 to 8.

According to some embodiments, the first plurality of mammalian cells comprises diseased cells.

According to some embodiments, the diseased cells are cancer cells.

According to some embodiments, the cancer is a cancer selected from the group consisting of colon cancer, colorectal cancer, lung cancer, prostate cancer, breast cancer, pancreatic cancer, liver cancer, kidney cancer, skin cancer, thyroid cancer, throat cancer, head or neck cancer, brain cancer, ovarian cancer, cervix cancer, spleen cancer, lymphoid cancer and hematopoietic cancer.

According to some embodiments, the mammalian cells are human cells.

According to some embodiments, the first plurality of mammalian cells, the second plurality of human cells or both are comprised in a composition which comprises an exogenous matrix material.

According to some embodiments, the composition comprises HA or Matrigel.

According to some embodiments, the composition comprises exogenous angiogenic growth factors.

According to some embodiments, the growth factors comprise bFGF and VEGF.

According to some embodiments, the placing a second plurality of mammalian cells is performed on embryo development day 8 to 10.

According to some embodiments, the immune cells comprise peripheral blood mononuclear cells (PBMCs).

According to some embodiments, the first plurality of mammalian cells and the second plurality of mammalian cells are derived from the same subject.

According to another aspect, there is provided a xenograft egg model produced by a method of the invention.

According to another aspect, there is provided a method of screening a therapeutic agent, the method comprising:
a. providing a xenograft egg model of the invention;
b. contacting the first plurality of mammalian cells with the therapeutic agent;
c. analyzing at least one response parameter in the first plurality of mammalian cell; and
d. selecting a therapeutic agent that produces a desired change in the at least one response parameter;

thereby screening a therapeutic agent.

According to some embodiments, the mammalian cells of the first plurality comprise diseased cells, the at least one response parameter comprises a disease phenotype or number of disease cells and the desired change is a reduction in disease phenotype or number of disease cells.

According to some embodiments, the disease is cancer, and the therapeutic agent is an anticancer therapeutic.

According to some embodiments, the therapeutic is an immunotherapeutic.

According to some embodiments, the mammalian cells of the first plurality comprise cancer cells and the immunotherapeutic is selected from an immune checkpoint inhibitor, an antibody against a cancer surface antigen and a modified immune cell.

According to some embodiments, the modified immune cell is a CAR immune cells and the CAR immune cell is a part of the second plurality of mammalian cells.

According to some embodiments, the contacting the first plurality of mammalian cells with the therapeutic agent is performed on embryo development day 9 or 10.

According to some embodiments, the therapeutic agent is injected into a yolk sac of the fertilized egg.

According to some embodiments, the analyzing is performed on embryo development day 15 or beyond.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: A schematic illustration of the screening platform created for patient-specific cancer treatment.

FIG. 2: A schematic illustration of the experiment timeline, from fertilized egg to analysis of the anticancer therapy. In short, on embryo development days 1-2 (EDD=1-2), the fertilized eggs were incubated in an incubator on a moving tray. On EDD=3, the embryo and vasculature were identified, and 2 ml of albumen was pulled from the bottom part of the egg using a syringe, this resulted in lowering of the embryo and separation of the CAM membrane from the eggshell. Next, a small window was created at the top part of the eggshell and resealed with adhesive tape, and eggs were returned to the incubator for chick embryo development. On EDD=5-6, the eggs were irradiated (2.5 Gy, rate of 15/sec). On EDD=6-7, the CAM was activated (at the site of transplantation) and cancer cells in suspension (mixed with matrigel, 1:1 by volume) or biopsy/tissue samples (mixed with 0.8% Hyaluronic acid), both mixed with a mixture of VEGF:bFGF, were engrafted at the top part of the CAM. On EDD=8-9, the human immune system was reconstructed by supplying an effective number of immune cells in close proximity to the tumor (following CAM activation). On EDD=9-10, the tested anticancer drugs were applied to the eggs by injection into the yolk sac or topically to the CAM (in close proximity to the tumor) following CAM activation. From EDD=13 and on evaluation of the responsiveness of the tumor to treatment is carried out.

FIGS. 3A-C: Representative photographs of established 3D tumors derived from different human cancer cell lines: (3A) BT549 cells (breast cancer cells), (3B) HT29 cells (colorectal adenocarcinoma cells), and (3C) Raji cells (Burkitt Lymphoma cells) and KM-H2 cells (Hodgkin's lymphoma cells).

FIGS. 4A-C: Representative photographs of tumor growth using different types of matrices: (4A) 0.8% Hyaluronic acid; (4B) E-C-L Cell Attachment Matrix (entactin-collagen IV-laminin); and (4C) Matrigel.

FIGS. 5A-D: (5A) Histological sections of HT29-GFP derived xenograft. (5B) frozen sections and the HT29-GFP derived xenograft. (5C-D) (5C) IHC and (5D) western blot of CD24 expression.

FIGS. 6A-C: Live imaging, using an In Vivo Imaging System (IVIS) device, of tumor engraftments onto CAM, showing (6A) a late stage of development and (6B-C) two early stages of development (24 and 48 hours after cell engraftment).

FIGS. 7A-G: Representative photographs of (7A-F) established 3D tumors from human-derived specimens: (7A) Chronic lymphocytic leukemia (CLL), (7B) colon adenoma (pre-malignant stage), (7C) ovarian cancer, (7D) uterine cancer, (7E) breast cancer, (7F) vulva cancer, and of (7G) serially passed HT29 cells.

FIGS. 8A-B: Bar chars of tumor weight (8A) in response to chemotherapeutic drugs or (8B) showing tumor heterogeneity in response to paclitaxel.

FIGS. 9A-E: Representative histograms showing (9A-B) mouse CD45+ cells in eggs that received (9A) 0 or 1 Gy of radiation, (9B) 0 or 2.5 Gy of radiation, (9C-D) human CD45+ cells in eggs that (9C) did not receive or (9D) did receive human immune cell transplant, and (9E) human CD8 positive T cells in transplanted eggs.

FIGS. 10A-C: (10A-B) Bar charts of tumor weight in eggs with reconstituted human immune systems (10A) exposed to anti-CD24 immunotherapy or to (10B) CAR-T therapy. (10C) Representative imagine of CAM models used in 10B.

FIGS. 11A-D: Bar charts of (11A, 11C) luminescence and (11B, 11D) tumor weight in (11A-B) MSI-high colorectal cancer CAM models and (11C-D) MSI-low colorectal CAM models treated with control or Keytruda in combination with CAR-T therapy.

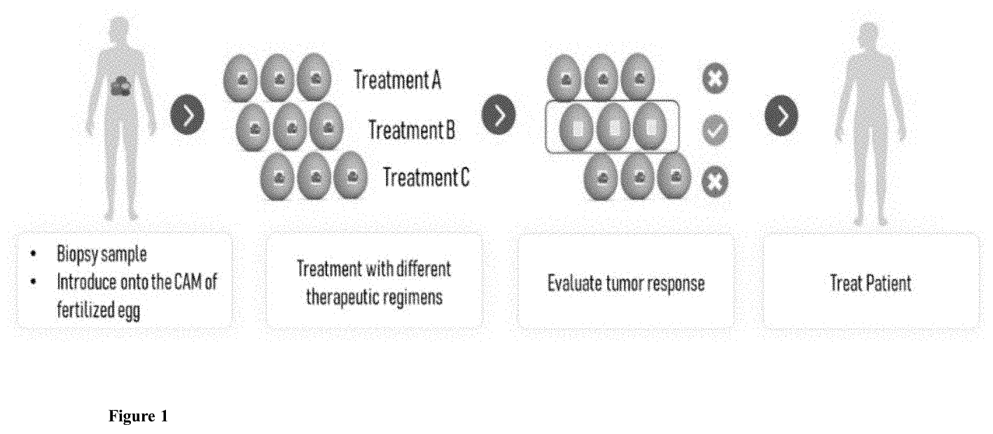

### DETAILED DESCRIPTION OF THE INVENTION

The present invention, in some embodiments, provides xenograft egg models comprising a fertilized non-mammalian egg comprising an ablated immune system, a first plurality of mammalian cells and a second plurality of mammalian cells, wherein the second plurality comprises immune cells are provided. Methods of producing the xenograft egg model as well as using the xenograft egg model for screening are also provided.

Predicting the clinical response to anti-cancer therapies is of great importance in cancer treatment. The use of “personalized medicine” aims to increase target interference, while maximizing benefit and minimizing harmful events.

The invention is based at least in part on the surprising generation of a xenograft egg model that is superior in its ability to test immunotherapies on patient specific samples. This allows for the personalized treatment selection of immunotherapies. This model utilizes a chick egg which is completely ablated of the chicken immune system and is manipulated to comprise disease cells along with immune cells from the same species, indeed from the same subject. This model enables the testing various cancer therapies, including immunotherapies, which require the immune system for their activity. The removal of the egg immune system ensures that all results are specifically caused by the therapeutic. Furthermore, the immune-ablated egg enables elongation of the window in which the egg model system can be used to beyond day 15 when normally the egg's immune system would be functional. This is of great importance when studying immunotherapies. The addition of immune cells from the subject requires an extra couple of days of culture and reduces the window in which the effects of the therapeutics can be monitored. In the complete absence of the chicken immune system the therapeutic effects can be monitored right up until hatching. This extended window is essential for testing immunotherapies and especially combined immunotherapies.

By a first aspect, there is provided a xenograft egg model comprising:
a. a fertilized egg;
b. a first plurality of explant cells; and
c. a second plurality of explant cells, comprising immune cells.

By another aspect, there is provided a method of producing a xenograft egg model, the method comprising:
a. providing a fertilized egg;
b. irradiating the fertilized egg;
c. placing a first plurality of explant cells in the egg; and
d. placing a second plurality of explant cells comprising immune cells in the egg;

thereby producing a xenograft egg model.

By another aspect, there is provided a xenograft egg model produced by a method of the invention.

As used herein the term “xenograft” as used herein refers to a cell or tissue transplant from a species different from the recipient organism (i.e., egg).

In some embodiments, the egg is a viable egg. In some embodiments, the egg is a fertilized egg. In some embodiments, the egg is a non-mammalian egg. In some embodiments, the egg is a non-human egg. In some embodiments, the egg is from a different species than the first plurality of cells. In some embodiments, the egg is from a different species than the second plurality of cells. In some embodiments, the egg is an ex vivo egg. In some embodiments, the egg is an egg that reaches maturity ex vivo. In some embodiments, the egg comprises a shell. In some embodiments, the egg comprises a hard eggshell. In some embodiments, the shell is a physical barrier. In some embodiments, the shell allows transfer of gasses to the egg's interior.

In some embodiments, the egg is an avian egg. In some embodiments, the egg is a reptilian egg. Exemplary avian eggs include, but are not limited to, a chicken, a turkey, a duck, a goose, a quail, a pheasant, a grouse, an ostrich, an emu, a cassowary or a kiwi egg. Exemplary reptilian eggs include, but are not limited to, lizard, turtle and crocodile eggs. In some embodiments, the avian egg is selected from the group consisting of a chicken egg, a turkey egg, a duck egg and a goose egg. In some embodiments, the egg is a chicken egg.

In some embodiments, the egg comprises vasculature. In some embodiments, the vasculature is non-mammalian vasculature. In some embodiments, the egg comprises a chorioallantoic membrane (CAM). In some embodiments, the CAM comprises vasculature. In some embodiments, the vasculature comprises the CAM is some embodiments, the CAM is the vasculature. In some embodiments, the vasculature supports the growth of an embryo in the egg.

In some embodiments, the egg is immunodeficient. As used herein, the term “immunodeficient” refers to an egg which lacks all or part of a functional immune system. In some embodiments, the immune system is a humoral immune system. In some embodiments, the immune system is an adaptive immune system. In some embodiments, the immune system is an innate immune system. In some embodiments, immunodeficient does not comprise an egg which has not yet developed an immune system. In some embodiments, immunodeficient comprises an egg that cannot form a fully functional immune system. In some embodiments, immunodeficient comprises an egg that cannot form a function immune system. In some embodiments, immunodeficient comprises an egg that has a decreased number of immune cells. In some embodiments, immune cells are CD45 positive cells. In some embodiments, immune cells are CD8 positive immune cells. In some embodiments, the decreased is as compared to a healthy egg. In some embodiments, decreased is as compared to a non-immunodeficient egg. In some embodiments, decreased is a decrease of at least 50, 60, 70, 75, 80, 85, 90, 92, 95, 97, 99 or 100%. Each possibility represents a separate embodiment of the invention. In some embodiments, decreased is a decrease of at least 50%. In some embodiments, decreased is a decrease of at least 75%. In some embodiments, decreased is a decrease of at least 90%. In some embodiments, decreased is a decrease of at least 95%.

In some embodiments, the egg comprises an ablated immune system. In some embodiments, immunodeficient comprises an ablated immune system. In some embodiments, ablated is partially ablated. In some embodiments, ablated is substantially ablated. In some embodiments, ablated is completely ablated. In some embodiments, an ablated immune system comprises reduced number of CD45 positive cells. In some embodiments, an ablated immune system is substantially devoid of CD45 positive cells. In some embodiments, substantially is at least 90%. In some embodiments, substantially is at least 95%. In some embodiments, substantially is at least 97%. In some embodiments, substantially is at least 99%. In some embodiments, an ablated immune system is devoid of immune cells. In some embodiments, an ablated immune system is devoid of CD45 positive cells. In some embodiments, the egg is ablated from its own immune system. In some embodiments, the egg is ablated of a non-mammalian immune system. In some embodiments, the egg is ablated of an endogenous immune system.

In some embodiments, the egg remains ablated of an immune system beyond the developmental day at which a natural immune system forms. In some embodiments, the egg remains ablated beyond day 13, 14, 15, 16, 17, 18, 19, 20 or 21 of development. Each possibility represents a separate embodiment of the invention. In some embodiments, the egg remains ablated beyond day 15 of development. In some embodiments, the egg remains ablated beyond day 13 of development. In some embodiments, the egg remains ablated beyond day 16 of development. In some embodiments, remaining ablated comprises remaining depleted of CD45 positive cells. In some embodiments, remaining ablated comprises remaining substantially devoid of CD45 positive cells. In some embodiments, remaining ablated comprises remaining devoid of CD45 positive cells.

For example, the immunodeficient egg may be characterized by a lack of functional immune cells. In some embodiments, an immune cell is selected from a T cell, a B cell, a macrophage and a neutrophil. In some embodiments, the immunodeficiency is present until hatching. In some embodiments, the immunodeficiency is permanent.

Immunodeficiency/ablation of the immune system of the egg may be generated by any external manipulation known in the art for suppressing functionality of an immune system. In some embodiments, immunodeficiency is induced by irradiation of the fertilized egg. In some embodiments, the irradiation is non-lethal irradiation. In some embodiments, the immunodeficiency/ablation is produced by irradiation of the egg. For example, by sub-lethal irradiation of the recipient egg with high frequency electromagnetic radiation, e.g., gamma radiation or x-rays, or proton therapy. Additionally, or alternatively, the egg may be treated with a radiomimetic drug such as busulfan or nitrogen mustard. In some embodiment, the irradiation is gamma radiation. In some embodiment, the irradiation is x-ray irradiation. Irradiation is well known in the art and any method or apparatus known may be used for this irradiation. In some embodiment, the immunodeficiency does not affect viability of the egg. In some embodiments, ablation does not affect viability of the egg.

According to one embodiment, the irradiation comprises a single or fractionated irradiation dose within the range of 0.5-1 Gray (Gy), 0.5-1.5 Gy, 0.5-2.5 Gy, 0.5-5 Gy, 0.5-7.5 Gy, 0.5-10 Gy, 0.5-15 Gy, 1-1.5 Gy, 1-2 Gy, 1-2.5 Gy, 1-3 Gy, 1-3.5 Gy, 1-4 Gy, 1-4.5 Gy, 1-1.5 Gy, 1-7.5 Gy, 1-10 Gy, 2-3 Gy, 2-4 Gy, 2-5 Gy, 2-6 Gy, 2-7 Gy, 2-8 Gy, 2-9 Gy, 2-10 Gy, 3-4 Gy, 3-5 Gy, 3-6 Gy, 3-7 Gy, 3-8 Gy, 3-9 Gy, 3-10 Gy, 4-5 Gy, 4-6 Gy, 4-7 Gy, 4-8 Gy, 4-9 Gy, 4-10 Gy, 5-6 Gy, 5-7 Gy, 5-8 Gy, 5-9 Gy, 5-10 Gy, 6-7 Gy, 6-8 Gy, 6-9 Gy, 6-10 Gy, 7-8 Gy, 7-9 Gy, 7-10 Gy, 8-9 Gy, 8-10 Gy, 10-12 Gy or 10-15 Gy. Each possibility represents a separate embodiment of the invention. In some embodiment, the irradiation comprises an irradiation dose of 2 to 5 Gy. In some embodiment, the irradiation comprises an irradiation dose of 1.5 to 3.5 Gy. In some embodiment, the irradiation comprises an irradiation dose of 1.5 to 4 Gy. In some embodiment, the irradiation comprises an irradiation dose of 2 to 4 Gy. In some embodiments, the irradiation comprises an irradiation dose of at least 2 Gy. In some embodiments, the irradiation comprises an irradiation dose of at least 2.5 Gy. In some embodiments, the irradiation comprises an irradiation dose of at most 3 Gy. In some embodiments, the irradiation comprises an irradiation dose of at most 4 Gy. In some embodiments, the irradiation comprises an irradiation dose of at most 5 Gy. In some embodiments, the irradiation comprises an irradiation dose of at most 6 Gy. In some embodiments, the irradiation comprises an irradiation dose of at most 7 Gy. In some embodiments, the irradiation comprises an irradiation dose of at most 10 Gy. In some embodiment, the irradiation comprises an irradiation dose of about 2.5 Gy. In some embodiment, the irradiation comprises an irradiation dose of 2.5 Gy.

In some embodiment, irradiation is affected at a rate of about 1-30, 5-30, 10-30, 15-30, 1-25, 5-25, 10-25, 15-25, 1-20, 5-20, 10-20, 15-20, 1-15, 5-15 or 10-15 rad/sec. Each possibility represents a separate embodiment of the invention. In some embodiment, irradiation is affected at a rate of about 10-20 rad/sec. In some embodiment, irradiation is affected at a rate of about 15 rad/sec. In some embodiment, irradiation is affected at a rate of 15 rad/sec. In some embodiments, the irradiating is performed on embryo development day 4 to 7. In some embodiments, the irradiating is performed on embryo development day 4. In some embodiments, the irradiating is performed on embryo development day 4-6. In some embodiments, the irradiating is performed on embryo development day 4-5. In some embodiments, the irradiating is performed on embryo development day 5-7. In some embodiments, the irradiating is performed on embryo development day 5-6. In some embodiments, the irradiating is performed on embryo development day 6-7. In some embodiments, the irradiating is performed by embryo development day 7. In some embodiments, the irradiating is performed before initial formation of the egg immune system. In some embodiments, the irradiating is performed before embryo development day 8.

In some embodiments, the first plurality of cells are mammalian cells. In some embodiments, the explant cells are mammalian cells. In some embodiments, the mammal is a human. In some embodiments, the mammalian cells are from a patient. In some embodiments, the mammalian cells of the first plurality are disease cells. In some embodiments, the mammalian cells of the first plurality are diseased cells. In some embodiments, the disease is a proliferative disease. In some embodiments, the disease cells are malignant. In some embodiments, the disease is cancer. In some embodiments, the disease cells are cells with a disease for which a therapeutic is to be tested.

In some embodiments, the cancer is a hematopoietic cancer. In some embodiments, the cancer is a blood cancer. In some embodiments, the cancer is CLL. In some embodiments, the cancer is ALL. In some embodiments, the cancer is lymphoma. In some embodiments, the lymphoma is Burkitt lymphoma. In some embodiments, the lymphoma is Hodgkin's lymphoma. In some embodiments, the lymphoma is non-Hodgkin's lymphoma. In some embodiments, the cancer is ovarian cancer. In some embodiments, the cancer is breast cancer. In some embodiments, the breast cancer is triple-negative breast cancer. In some embodiments, the cancer is colorectal cancer. In some embodiments, the cancer is adenocarcinoma. In some embodiments, the cancer is colon cancer. In some embodiments, the cancer is ovarian cancer. In some embodiments, the cancer is uterine cancer. In some embodiments, the cancer is vulval cancer. In some embodiments, the cancer is metastatic. In some embodiments, the cancer is non-metastatic. In some embodiments, the cancer is selected from the group consisting of colon cancer, colorectal cancer, lung cancer, prostate cancer, breast cancer, pancreatic cancer, liver cancer, kidney cancer, skin cancer, thyroid cancer, throat cancer, head and neck cancer, brain cancer, ovarian cancer, cervix cancer, spleen cancer, lymphoid cancer and hematopoietic cancer.

According to a specific embodiment, the mammalian cells or tissues are of a cancer selected from the group consisting of colon cancer, colorectal cancer, lung cancer, prostate cancer, breast cancer, pancreatic cancer, liver cancer, kidney cancer, skin cancer, thyroid cancer, throat cancer, head or neck cancer, brain cancer, ovarian cancer, cervix cancer, spleen cancer, myeloid cancer, lymphoid cancer and hematopoietic cancer.

Examples of lymphoid, hematopoietic and myeloid cancers include, but are not limited to, leukemia [e.g., acute lymphatic, acute lymphoblastic leukemia (ALL), acute lymphoblastic pre-B cell, acute lymphoblastic T cell leukemia, acute-megakaryoblastic, monocytic, acute myelogenous, acute myeloid, acute myeloid with eosinophilia, B cell, basophilic, chronic myeloid, chronic, B cell, eosinophilic, Friend, granulocytic or myelocytic, acute myelocytic leukemia (AML) or chronic myelocytic leukemia (CML), hairy cell, lymphocytic, megakaryoblastic, monocytic, monocytic-macrophage, myeloblastic, myeloid, myelomonocytic, plasma cell, pre-B cell, promyelocytic, subacute, T cell, lymphoid neoplasm, predisposition to myeloid malignancy, acute nonlymphocytic leukemia, acute nonlymphoblastic leukemia (ANLL), T-cell acute lymphocytic leukemia (T-ALL) and B-cell chronic lymphocytic leukemia (B-CLL)]; lymphoma (e.g., Hodgkin's disease, non-Hodgkin's lymphoma, follicular lymphoma, B cell lymphoma, Burkitt's Lymphoma, diffuse large B cell lymphoma (DLBCL), mantle cell lymphoma (MCL), T cell lymphoma, thymic lymphoma, cutaneous T cell lymphoma, histiocytic lymphoma, lymphoblastic lymphoma); myeloid (e.g. acute myeloid leukemia (AML); myelodysplastic syndromes (MDS), chronic myeloid leukemia (CML) or other myeloproliferative diseases (e.g., osteomyelofibrosis, polycythemia vera and essential thrombocythemia)).

In some embodiments, the first plurality of cells is in contact with vasculature. In some embodiments, the vasculature is the egg's vasculature. In some embodiments, the vasculature is the CAM. In some embodiments, the first plurality of cells is in contact with the CAM. In some embodiments, the CAM is the upper CAM. Upper refers to the upper part of the egg, above the growing fetus. In some embodiments, the first plurality of cells is located apically on the CAM.

In some embodiments, the second plurality of cells is in contact with vasculature. In some embodiments, the vasculature is the egg's vasculature. In some embodiments, the vasculature is the CAM. In some embodiments, the second plurality of cells is in contact with the CAM. In some embodiments, the CAM is the upper CAM. In some embodiments, the second plurality of cells is located apically on the CAM.

In some embodiments, the CAM is activated before placing. In some embodiments, the method comprises activating the CAM before placing. In some embodiments, before is immediately before. In some embodiments, before is at the same time. In some embodiments, activation is before placing the first plurality. In some embodiments, activation is before placing the second plurality. In some embodiments, activating comprises contact with trypsin. In some embodiments, activating comprises physical agitation. Agitation can be performed with a Q-tip, pick or the like. Any method of activation of the CAM may be employed. In some embodiments, activation is activation of angiogenesis. In some embodiments, angiogenesis is angiogenesis toward the explant cells.

In some embodiments, the first plurality of cells is devoid of immune cells. In some embodiments, the first plurality of cells comprises tumor resident immune cells. In some embodiments, the first plurality of cells is suspension of cells. In some embodiments, the first plurality of cells is a tissue. In some embodiments, the first plurality of cells is a fragment of tissue. In some embodiment, the suspension of cells is produced from a tissue. In some embodiments, the tissue is a tumor. In some embodiments, the fragment is a biopsy. In some embodiments, the first plurality of cells are cells of a cell line. In some embodiments, the cell line is a human cell line. In some embodiments, the first plurality of cells are primary cells. In some embodiments, the primary cells are from primary cell culture. In some embodiments, the primary cells are directly from a subject. In some embodiments, the cell line is a cancer cell line. In some embodiments, the cancer cell line is from the same type of cancer as a cancer of the subject.

Exemplary tissues or organs which may be transplanted or dissociated into a single cell solution and transplanted according to the present teachings include, but are not limited to, liver, pancreas, spleen, kidney, heart, lung, skin, intestine, colon, breast, ovarian, cervix, prostate, thyroid, brain and lymphoid/hematopoietic tissues (e.g., lymph node, Peyer's patches thymus or bone marrow). Exemplary cells which may be transplanted according to the present teachings include, but are not limited to, immature hematopoietic cells (including stem cells), cardiac cells, hepatic cells, pancreatic cells, spleen cells, pulmonary cells, brain cells, nephric cells, intestine/gut/colon cells, ovarian cells, cervix cells, prostate cells, thyroid cells, breast cells, skin cells.

In some embodiments, the placing of the first plurality of cells is performed on embryo development day 5-8. In some embodiments, the placing of the first plurality of cells is performed on embryo development day 6-8. In some embodiments, the placing of the first plurality of cells is performed on embryo development day 7-8. In some embodiments, the placing of the first plurality of cells is performed on embryo development day 6-7. In some embodiments, the placing of the first plurality of cells is performed on embryo development day 6. In some embodiments, the placing of the first plurality of cells is performed on embryo development day 7. In some embodiments, the placing of the first plurality of cells is performed on embryo development day 8.

In some embodiments, the placing of the first plurality of cell is before the placing of the second plurality of cells. In some embodiments, the placing of the first plurality of cells and the second plurality of cells is separated by a sufficient time for taking and growth of the first plurality of cells. In some embodiments, the placings are separated by at least 1 day. In some embodiments, the placings are separated by at least 2 days. In some embodiments, the placings are separated by 1-2 days. In some embodiments, the placing of the second plurality of cells is performed on embryo development day 7-11. In some embodiments, the placing of the first plurality of cells is performed on embryo development day 7-10. In some embodiments, the placing of the first plurality of cells is performed on embryo development day 8-11. In some embodiments, the placing of the first plurality of cells is performed on embryo development day 8-10. In some embodiments, the placing of the first plurality of cells is performed on embryo development day 8-9. In some embodiments, the placing of the first plurality of cells is performed on embryo development day 9-10. In some embodiments, the placing of the first plurality of cells is performed on embryo development day 8. In some embodiments, the placing of the first plurality of cells is performed on embryo development day 9. In some embodiments, the placing of the first plurality of cells is performed on embryo development day 10.

In some embodiments, the cells of the first plurality and the cells of the second plurality are from the same species. In some embodiments, the cells of the first plurality and the cells of the second plurality are from the same subject. In some embodiments, the cells of the first plurality and the cells of the second plurality are derived from the same subject. In some embodiments, the cells of the first plurality were extracted from a subject. In some embodiments, the cells of the second plurality were extracted from a subject. In some embodiments, the method further comprises extracting the first plurality from a subject. In some embodiments, the method further comprises extracting the second plurality from a subject. In some embodiments, the method further comprises receiving the first plurality from a subject. In some embodiments, the method further comprises receiving the second plurality from a subject. In some embodiments, the receiving is receiving a sample and the sample comprises the cells of the plurality.

In some embodiments, the first plurality and the second plurality are proximal to each other. In some embodiments, the first plurality and the second plurality are close to each other. In some embodiments, the first plurality and the second plurality are within 10, 7, 5, 4, 3, 2, 1, 0.9, 0.8, 0.7, 0.6, 0.5, 0.4, 0.3, 0.2 or 0.1 cm of each other. Each possibility represents a separate embodiment of the invention. In some embodiments, the first plurality and the second plurality are within 1 cm of each other. In some embodiments, within a distance is separated by a distance of less than that amount. In some embodiments, the first plurality and the second plurality are not in direct contact. In some embodiments, the first plurality and the second plurality are within the same composition. In some embodiments, the first plurality and the second plurality are not within physical contact. In some embodiments, the first plurality and the second plurality are not within direct physical contact. It will be understood by a skilled artisan that the immune cells of the second plurality are not merely tumor resident immune cells within the first plurality, but rather are separate immune cells which were separately prepared and add at a separate time.

In some embodiments, the second plurality comprises immune cells. In some embodiments, the immune cells are mammalian immune cells. In some embodiments, the immune cells are form a subject. In some embodiments, the presence of the immune cells reconstitutes the ablated/missing immune system. In some embodiments, the egg is reconstituted with non-mammalian immune cells. As used herein, the term “reconstituted with immune cells” or “immune reconstitution” refers to restoring a functional immune system, in full or in part, to the egg (i.e., with mammalian immune cells).

The egg may be reconstituted with, or by, mammalian hematopoietic stem cells (HSCs) or by mammalian-derived immune cells (e.g., peripheral blood mononuclear cells, i.e., PBMCs). In some embodiments, the immune cells are HSCs. In some embodiments, the immune cells comprise HSCs. In some embodiments, the immune cells are PBMCs. In some embodiments, the immune cells comprise PBMCs. In some embodiments, the immune cells comprise CD45 positive cells. In some embodiments, the immune cells comprise stem cells. In some embodiments, the immune cells comprise progenitor cells. In some embodiments, the immune cells are sufficient to reconstitute the immune system. In some embodiments, the immune cells comprise T cells. In some embodiments, the T cells are CD8 positive T cells. In some embodiments, the T cells are CD4 positive T cells. In some embodiments, the immune cells comprise B cells. In some embodiments, the immune cells comprise macrophages. In some embodiments, the immune cells comprise cytotoxic immune cells. In some embodiments, the immune cells comprise natural killer cells (NK). In some embodiments, the immune cells are capable of migrating to the first plurality of cells. In some embodiments, the immune cells are capable of migrating into the vasculature. In some embodiments, the immune cells are capable of homing to the first plurality.

As used herein, HSCs (e.g., human HSCs) are self-renewing stem cells that, when engrafted into a recipient, can “repopulate” or “reconstitute” the hematopoietic system of the recipient (e.g., the immune deficient egg) and sustain (e.g., long term) hematopoiesis in the recipient. The hematopoietic system refers to the organs and tissue involved in the production of the blood cell lineages (e.g., bone marrow, spleen, tonsils, lymph nodes). HSCs are multipotent stem cells that give rise to (differentiate into) blood cell types including myeloid cell lineages (e.g., monocytes and macrophages, neutrophils, basophils, eosinophils, erythrocytes, megakaryocytes/platelets, dendritic cells) and lymphoid cell lineages (e.g., T-cells, B-cells, NK-cells).

HSCs are found in bone marrow such as in femurs, hip, ribs, sternum, and other bones of a mammal (e.g., humans, primates, pigs, mice, etc.). Other sources of HSCs for clinical and scientific use include umbilical cord blood, placenta, fetal liver, mobilized peripheral blood, non-mobilized (or unmobilized) peripheral blood, fetal liver, fetal spleen, embryonic stem cells, and aorta-gonad-mesonephros (AGM), or a combination thereof.

As will be understood by persons of skill in the art, mobilized peripheral blood refers to peripheral blood that is enriched with HSCs (e.g., CD34+ cells). Administration of agents such as G-CSF mobilizes stem cells from the bone marrow to the peripheral circulation.

In some embodiments, PBMCs comprise a mixture of immune cells including, but not limited, to lymphocytes (T cells, B cells, NK cells) and monocytes. In some embodiments, the immune cells are unmodified immune cells. In some embodiments, the immune cells are modified immune cells. In some embodiments, modified is engineered. In some embodiments, the immune cells are expanded in culture. In some embodiments, the immune cells are primary immune cells. In some embodiments, the immune cells are from a subject. In some embodiments, the immune cells are from a subject suffering from cancer. In some embodiments, the first plurality and the second plurality of syngeneic to each other.

In some embodiments, the immune cells have been enhanced. In some embodiments, cytotoxicity is enhanced. In some embodiments, the immune cells are chimeric antigen receptor (CAR) immune cells. In some embodiments, the CAR cells are CAR-T cells. In some embodiments, the CAR cells are CAR-NK cells.

The immune cells used for immune reconstitution according to the teachings of the invention can be obtained from a single donor or multiple donors. In addition, the immune cells used for immune reconstitution according to the teachings of the invention and can be freshly isolated, cryopreserved, or a combination thereof.

In some embodiments, the mammal is a human. In some embodiments, the mammal is a non-human. In some embodiments, the mammalian cells or tissues are obtained from a non-human organism. Exemplary non-human mammals include, but are not limited to, mice, rats, hamsters, gerbils, guinea pigs, rabbits, cats, dogs, pigs, cows, goats, sheep, horses, donkeys, deer, antelopes, elephants, camels, llamas, primates (e.g., monkeys, apes), and the like.

In some embodiments, the first plurality of cells is comprised in a composition. In some embodiments, the second plurality of cells is comprised in a composition. In some embodiments, the composition comprises a matrix material. In some embodiments, the matrix material is an exogenous matrix material. In some embodiments, the matrix material is in organic. In some embodiments, the matrix material is artificial. In some embodiments, the matrix material is extracellular matrix. In some embodiments, the composition comprises hyaluronic acid (HA). In some embodiments, the matrix material comprises HA. In some embodiments, the composition comprises Matrigel. In some embodiments, the matrix material comprises Matrigel. In some embodiments, the matrix material is Matrigel. In some embodiments, cells are comprised in Matrigel. In some embodiments, tissue is comprised in a composition comprising HA. In some embodiments, the matrix material is capable of supporting cell growth in the egg.

As used herein, the “matrix” refers to a complex non-cellular three-dimensional macromolecular network. In some embodiments, the matrix comprises a plurality of collagens, proteoglycans/glycosaminoglycans, elastin, fibronectin, laminins, heparin sulfate proteoglycans, entactin/nidogens, and/or other glycoproteins. These molecules are typically secreted locally by cells and remain closely associated with them to provide structural, adhesive and biochemical signaling support.

Regardless of the matrix material used, angiogenic growth factors are used in the composition to support growth of the cells or tissues in the egg. In some embodiments, the composition comprises growth factors. In some embodiments, the matrix material comprises growth factors. In some embodiments, the growth factors are angiogenic growth factors. Exemplary angiogenic growth factors include, but are not limited to, basic fibroblast growth factor (bFGF), platelet-derived endothelial cell growth factor (PD-ECGF), platelet-derived growth factor (PDGF), vascular endothelial growth factor (VEGF), placental growth factor (P1GF), granulocyte-colony stimulating factor (G-CSF or GCSF), granulocyte-macrophage colony-stimulating factor (GM-CSF), epidermal growth factor (EGF), nerve growth factor (NGF). In some embodiment, the growth factors are selected from VEGF and bFGF. In some embodiments, the growth factors are VEGF and bFGF.

According to a specific embodiment, the matrix material, the Hyaluronic acid and the angiogenic growth factors used in the composition are exogenous with respect to the egg, i.e., originating or produced from outside of the egg. According to a specific embodiment, the matrix material, the Hyaluronic acid and the angiogenic growth factors used in the composition are not naturally secreted by the implanted cells or tissues.

In some embodiments, the egg comprises an eggshell. In some embodiments, the method comprises opening the eggshell. In some embodiments, the method comprises removing at least a part of egg albumen. In some embodiments, a part is about 2 ml. In some embodiments, a part is 2 ml. In some embodiments, a part is at least 2 ml. In some embodiments, a part is 1-3 ml. In some embodiments, a part is 1.5-2.5 ml. In some embodiments, the albumen is removed from a bottom region of the egg. In some embodiments, removal separates a CAM from the eggshell. In some embodiments, the removal is configured to separate a CAM from the eggshell. In some embodiments, the removal thereby separates a CAM from the eggshell. In some embodiments, the removing the albumen is performed at embryo development day 1-3. In some embodiments, the removing the albumen is performed at embryo development day 2-3. In some embodiments, the removing the albumen is performed at embryo development day 3. In some embodiments, the removing the albumen is performed not after embryo development day 3. In some embodiments, the removing the albumen is performed before fusion of the CAM to the eggshell.

In some embodiments, the method comprises creating an aperture in the eggshell. In some embodiments, aperture is in a top region of the eggshell. In some embodiments, the aperture exposes at least a portion of the CAM. In some embodiments, the aperture gives access to the CAM. If the cells of the first and second plurality can be contacted without an aperture (i.e., by injection) then an aperture may not be necessary. It is sufficient that the cells can be provided to the vasculature (e.g., the CAM). In some embodiments, the creating an aperture is performed at embryo development day 1-3. In some embodiments, the creating an aperture is performed at embryo development day 2-3. In some embodiments, the creating an aperture is performed at embryo development day 3. In some embodiments, the creating an aperture is performed not after embryo development day 3. In some embodiments, the creating an aperture is performed before fusion of the CAM to the eggshell. In some embodiments, the method comprises resealing the aperture. In some embodiments, resealing segregates the egg inside the eggshell from an environment outside the eggshell. In some embodiments, segregates is physically segregates.

By another aspect, there is provided a method of screening a therapeutic agent, the method comprising:
a. providing a xenograft egg model of the invention;
b. contacting the first plurality of mammalian cells with the therapeutic agent; and
c. analyzing at least one response parameter in the first plurality of cells;

thereby screening a therapeutic agent.

In some embodiments, the method further comprises selecting a therapeutic agent that produces a desired change in the at least one response parameter. In some embodiments, the first plurality of cells comprises disease cells and the at least one response parameter comprises a disease phenotype. In some embodiments, the response parameter is the number of cells. In some embodiments, the first plurality of cells comprises disease cells and the at least one response parameter comprises the number of disease cells. In some embodiments, the response parameter is proliferation. In some embodiments, a disease phenotype is proliferation. In some embodiments, a disease phenotype is expression of a disease marker. In some embodiments, the marker is a surface marker. In some embodiments, the marker is a protein. In some embodiments, the marker is an RNA. In some embodiments, the disease phenotype is tumor size. In some embodiments, the disease phenotype is lack of differentiation. In some embodiments, a disease marker is inflammation. In some embodiments, inflammation comprises expression of a proinflammatory cytokine.

In some embodiments, decrease in the parameter is a desired result. In some embodiments, increase in the parameter is a desired result. In some embodiments, increase is as compared to before the contacting. In some embodiments, increase is as compared to the xenograft egg model without contacting with the therapeutic. In some embodiments, a decrease in a disease phenotype is desired. It will be understood by a skilled artisan that the output the therapeutic is designed to produce will be the desired output. In some embodiments, the desired result or desired output is the desired change. In some embodiments, the change is the change in the parameter. In some embodiments, the desired change in a disease phenotype is reduction.

In some embodiments, the analyzing is measuring. In some embodiments, measuring is measuring the parameter. In some embodiments, the analyzing is performed after contacting with the therapeutic. In some embodiments, the analyzing is performed a sufficient time after the contacting with the therapeutic to allow the therapeutic to exert its therapeutic effect. This sufficient time may be dependent on the therapeutic, the method of administration, the dosage, the number of immune cells present of a combination thereof. A major advantage of the xenograft of the invention is that with do developing egg immune system the effect of the therapy can be analyzed/measured all the way until the point of hatching, greatly expanding the window for this analyzing. In some embodiments, the analyzing is performed at least 1 day after placing the therapeutic agent. In some embodiments, the analyzing is performed at least 2 days after placing the therapeutic agent. In some embodiments, the analyzing is performed at least 3 days after placing the therapeutic agent. In some embodiments, the analyzing is performed at least 4 days after placing the therapeutic agent. In some embodiments, the analyzing is performed at least 5 days after placing the therapeutic agent. In some embodiments, the analyzing is performed on a day that when there would be an egg immune system had it not been ablated. In some embodiments, an egg immune system is a functional egg immune system. In some embodiments, functional is fully functional. In some embodiments, the analyzing is performed on embryo development day 13 or beyond. In some embodiments, the analyzing is performed on embryo development day 14 or beyond. In some embodiments, the analyzing is performed on embryo development day 15 or beyond. In some embodiments, the analyzing is performed on embryo development day 16 or beyond.

As used herein “therapeutic agent” refers to a therapeutically active ingredient such as a small molecule (e.g., chemotherapy), a toxin, a protein (e.g., an antibody), a lipid, a carbohydrate, a nucleic acid (e.g. a nucleic acid silencing agent, such as a siRNA, miRNA or antisense), or a combination of same.

In some embodiments, the disease is cancer, and the therapeutic agent is an anticancer agent. In some embodiments, the disease is cancer, and the therapeutic agent is an anticancer therapy. In some embodiment, the therapeutic agent is an immunotherapy. In some embodiments, an immunotherapy is an immunotherapeutic. As used herein, an “immunotherapy” is any treatment that makes use of the immune system to produce a therapeutic effect.

In some embodiments, the immunotherapy is an immune checkpoint inhibitor. In some embodiments, the immunotherapy is an antibody. In some embodiments, the antibody is a monoclonal antibody. Immune checkpoint inhibitors are well known in the art and any may be used. Immune checkpoint proteins include, for example, PD-1, PD-L1, PD-L2, CTLA4, 4-IBB, HVEM and others. In some embodiments, the antibody is an anti-PD-1 antibody.

In some embodiments, the antibody is an antibody against a disease cell surface antigen. In some embodiments, the antigen is a cancer antigen. In some embodiments, the antibody induces antibody-dependent cell cytotoxicity (ADCC). In some embodiments, the antibody induces complement-dependent cytotoxicity (CDC). In some embodiments, the antibody is an IgG1 or IgG3 antibody or a modified IgG2 or IgG4 antibody wherein the modification enhances ADCC, CDC or both.

In some embodiments, the immunotherapy is a transplanted immune cell. In some embodiments, the immune cell is unmodified. In some embodiments, the immune cell is modified. In some embodiments, the modification is activation. In some embodiments, the modification enhances cytotoxicity. In some embodiments, the modification is expression of a CAR. In some embodiments, the transplanted immune cell is adoptive immune cell therapy. In some embodiments, the transplanted cell is a CAR-T cell. In some embodiments, the transplanted cell is a CAR-NK cell.

As used herein, the terms “CAR-T cell” and “CAR-NK cell” refer to an engineered receptor which has specificity for at least one protein of interest (for example an immunogenic protein with increased expression following treatment with an epigenetic modifying agent) and is grafted onto an immune effector cell (a T cell or NK cell). In some embodiments, the CAR-T cell has the specificity of a monoclonal antibody grafted onto a T-cell. In some embodiments, the CAR-NK cell has the specificity of a monoclonal antibody grafted onto a NK-cell. In some embodiments, the T cell is selected from a cytotoxic T lymphocyte and a regulatory T cell.

CAR-T and CAR-NK cells and their vectors are well known in the art. Such cells target and are cytotoxic to the protein for which the receptor binds. In some embodiments, a CAR-T or CAR-NK cell targets at least one viral protein. In some embodiments, a CAR-T or CAR-NK cell targets a plurality of viral proteins. In some embodiments, a CAR-T or CAR-NK cell targets a viral protein with increased expression due to contact with an epigenetic modifying agent.

Construction of CAR-T cells is well known in the art. In one non-limiting example, a monoclonal antibody to a viral protein can be made and then a vector coding for the antibody will be constructed. The vector will also comprise a costimulatory signal region. In some embodiments, the costimulatory signal region comprises the intracellular domain of a known T cell or NK cell stimulatory molecule. In some embodiments, the intracellular domain is selected from at least one of the following: CD3Z, CD27, CD28, 4-1BB, OX40, CD30, CD40, PD-1, ICOS, lymphocyte function-associated antigen- 1 (LFA-1), CD2, CD 7, LIGHT, NKG2C, B7- H3, and a ligand that specifically binds with CD83. In some embodiments, the vector also comprises a CD3Z signaling domain. This vector is then transfected, for example by lentiviral infection, into a T-cell. In some embodiments, the immune cell is part of the second plurality of cells.

In some embodiments, the contacting with the therapeutic agent is performed after placing the second plurality of cells. In some embodiments, the contacting of the therapeutic agent is performed a sufficient time after the placing the second plurality of cells so as to allow the immune cells to migrate to the first plurality of cells. In some embodiments, a sufficient time is at least 1 day. In some embodiments, a sufficient time is at least 2 days. In some embodiments, a sufficient time is 1-2 days. In some embodiments, the contacting of the therapeutic agent is performed on embryo development day 8-12. In some embodiments, the contacting of the therapeutic agent is performed on embryo development day 8-11. In some embodiments, the contacting of the therapeutic agent is performed on embryo development day 8-10. In some embodiments, the contacting of the therapeutic agent is performed on embryo development day 9-12. In some embodiments, the contacting of the therapeutic agent is performed on embryo development day 9-11. In some embodiments, the contacting of the therapeutic agent is performed on embryo development day 9-10. In some embodiments, the contacting of the therapeutic agent is performed on embryo development day 10-12. In some embodiments, the contacting of the therapeutic agent is performed on embryo development day 10-11. In some embodiments, the contacting of the therapeutic agent is performed on embryo development day 8. In some embodiments, the contacting of the therapeutic agent is performed on embryo development day 9. In some embodiments, the contacting of the therapeutic agent is performed on embryo development day 10. In some embodiments, the contacting of the therapeutic agent is performed after embryo development day 8. In some embodiments, the contacting of the therapeutic agent is performed after embryo development day 9.

Exemplary immunotherapies include, but are not limited to, immune checkpoint inhibitors (e.g. Ipilimumab (Yervoy®), Nivolumab (Opdivo®), Pembrolizumab (Keytruda®)); monoclonal antibodies (e.g. Rituximab (Mabthera®), Cetuximab (Erbitux®), Trastuzumab (Herceptin®)), immune system modulators (e.g. cytokines, growth factors, such as e.g. Thalidomide (Thalomid®), Lenalidomide (Revlimid®), Pomalidomide(Pomalyst®), Imiquimod (Aldara®, Zyclara®)), treatment vaccines, such as e.g. antigen vaccines, whole cell vaccines, dendritic cell vaccines, DNA vaccines, such as e.g. the cell-based cancer immunotherapy Sipuleucel-T (APC8015, trade name Provenge®), and biopharmaceutical drugs such as e.g. T-VEC (Imlygic™).

According to one embodiment, the therapeutic agent is a chemotherapeutic agent. Chemotherapeutic agents include, but are not limited to, fluoropyrimidines; pyrimidine nucleosides; purine nucleosides; anti-folates, platinum agents; anthracyclines/anthracenediones; epipodophyllotoxins; camptothecins (e.g., Karenitecin); hormones; hormonal complexes; antihormonals; enzymes, proteins, peptides and polyclonal and/or monoclonal antibodies; immunological agents; vinca alkaloids; taxanes; epothilones; antimicrotubule agents; alkylating agents; antimetabolites; topoisomerase inhibitors; antivirals; and various other cytotoxic and cytostatic agents.

According to a specific embodiment, the chemotherapeutic agent includes, but is not limited to, abarelix, aldesleukin, aldesleukin, alemtuzumab, alitretinoin, allopurinol, altretamine, amifostine, anastrozole, arsenic trioxide, asparaginase, azacitidine, bevacuzimab, bexarotene, bleomycin, bortezomib, busulfan, calusterone, capecitabine, carboplatin, carmustine, celecoxib, cetuximab, cisplatin, cladribine, clofarabine, cyclophosphamide, cytarabine, dacarbazine, dactinomycin, actinomycin D, Darbepoetin alfa, Darbepoetin alfa, daunorubicin liposomal, daunorubicin, decitabine, Denileukindiftitox, dexrazoxane, dexrazoxane, docetaxel, doxorubicin, dromostanolone propionate, Elliott's B Solution, epirubicin, Epoetin alfa, erlotinib, estramustine, etoposide, exemestane, Filgrastim, floxuridine, fludarabine, fluorouracil 5-FU, fulvestrant, gefitinib, gemcitabine, gemtuzumabozogamicin, goserelin acetate, histrelin acetate, hydroxyurea, IbritumomabTiuxetan, idarubicin, ifosfamide, imatinibmesylate, interferon alfa 2a, Interferon alfa-2b, irinotecan, lenalidomide, letrozole, leucovorin, Leuprolide Acetate, levamisole, lomustine, CCNU, meclorethamine, nitrogen mustard, megestrol acetate, melphalan, L-PAM, mercaptopurine 6-MP, mesna, methotrexate, mitomycin C, mitotane, mitoxantrone, nandrolonephenpropionate, nelarabine, Nofetumomab, Oprelvekin, Oprelvekin, oxaliplatin, paclitaxel, palifermin, pamidronate, pegademase, pegaspargase, Pegfilgrastim, pemetrexed disodium, pentostatin, pipobroman, plicamycinmithramycin, porfimer sodium, procarbazine, quinacrine, Rasburicase, Rituximab, sargramostim, sorafenib, streptozocin, sunitinib maleate, tamoxifen, temozolomide, teniposide VM-26, testolactone, thioguanine 6-TG, thiotepa, thiotepa, topotecan, toremifene, Tositumomab, Trastuzumab, tretinoin ATRA, Uracil Mustard, valrubicin, vinblastine, vinorelbine, zoledronate and zoledronic acid. Additional antineoplastic agents include those disclosed in Chapter 52, Antineoplastic Agents (Paul Calabresi and Bruce A. Chabner), and the introduction thereto, 1202-1263, of Goodman and Gilman's “The Pharmacological Basis of Therapeutics”, Eighth Edition, 1990, McGraw-Hill, Inc. (Health Professions Division).

In some embodiments, the therapeutic agent is injected into a yolk sac of the egg. In some embodiments, injection to the yolk sac mimic intravenous administration. In some embodiments, the therapeutic agent is injected into vasculature. In some embodiments, the therapeutic agent is administered onto the CAM. In some embodiments, the therapeutic agent is administered proximal to the CAM. In some embodiments, the therapeutic agent is administered proximal to the first plurality of cells. In some embodiments, the therapeutic agent is administered proximal to the second plurality of cells.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### Examples

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Materials and Methods

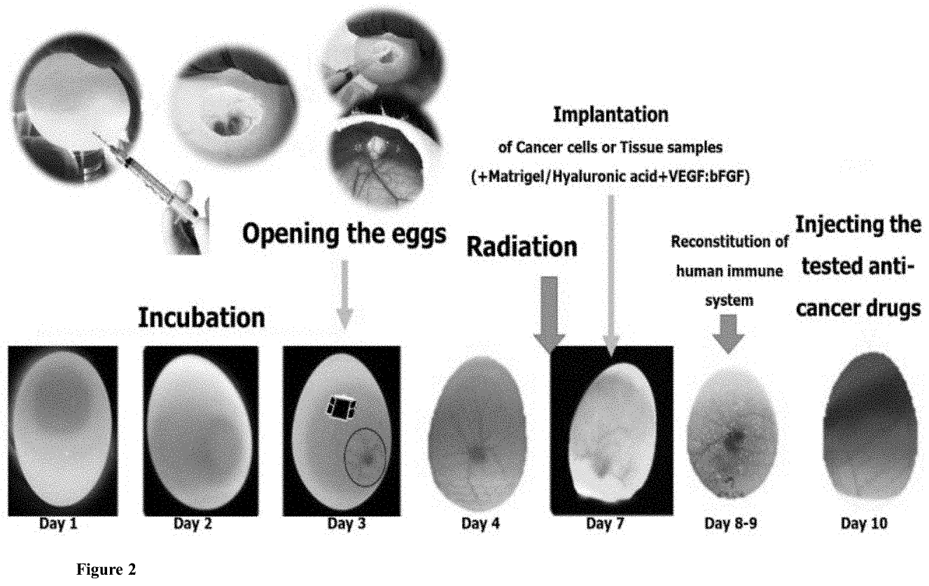

Egg Preparation and Tumor Cell Inoculation: An outline of the following procedure is provided in FIG. 2. Fertilized eggs were incubated for 72 days in an incubator (at 37° C., 75-90% humidity) on a moving tray, rotating the eggs 12 times a day. On embryo development day 3 (EDD=3), the embryo and vasculature were identified (using a flashlight in a dark room) and 2 ml of egg white (albumen) was pulled from the bottom part of the egg using a syringe, in order to lower the embryo, separate the CAM membrane from the eggshell, and generate space at the top part of the egg for introducing the cancer cells. Next, a small window was created at the top part of the eggshell and resealed with adhesive tape (in order to reduce dehydration and contamination), and eggs were returned to the incubator for chick embryo development.

On EDD=5 or 6 (i.e. 1 or 2 days prior to implantation of cancer cells), the eggs were irradiated (2.5 Gy, rate of 15/sec) in order to prevent the development of the chicken immune system. On EDD=6 or 7, the CAM was activated (at the site of transplantation) by gently touching the membrane with a Q-tip, thus increasing the propensity of the membrane to generate new vascularization toward the site of activation. Next, and immediately prior to addition of the cancer cells, 5 μl of 0.05% trypsin/0.5 mM EDTA was added to the site of transplantation in order to enhance the efficiency of tumor formation.

Next, cancer cells in suspension (3-5×10\hat{ }6 cells), mixed with matrix material (e.g. Matrigel) at a ratio of 1:1 by volume and a mixture of VEGF:basic FGF in a total volume of 30 μl, was engrafted at the top part of the CAM. Alternatively, biopsy/tissue samples were mixed with 0.8% Hyaluronic acid (HA) and a mixture of VEGF:basic FGF and engrafted at the top part of the CAM. Chick eggs were then resealed and returned to the incubator.

On EDD=8 or 9, the human immune system was introduced by supplying an effective number of immune cells. Specifically, peripheral blood mononuclear cells (PBMCs) were isolated from whole blood of healthy volunteers or from tested patients using Ficoll density gradient centrifugation. PBMCs were mixed with matrix material (e.g. Matrigel) (at a ratio of 3:1 cells to Matrigel by volume) and a mixture of VEGF:basic FGF. The immune cell mixture was engrafted at the top part of the CAM (in close proximity to the tumor) following CAM activation (as discussed above).

On EDD=9 or 10, the tested anti-cancer drugs were applied to the eggs by injection into the yolk sac or topically to the CAM (in close proximity to the tumor) following CAM activation (as discussed above). Evaluation of the responsiveness of the tumor to treatment, as compared to control untreated tissue, was performed on days 13-20.

Cell Lines: Cell lines used in the present study include: HT29 (colorectal adenocarcinoma), HCT116 (colon carcinoma), A549 (lung carcinoma), 1975 (lung carcinoma), DU145 (metastatic prostate cancer), PC3 (metastatic prostate cancer), BT549 (ductal carcinoma), 468 (triple negative breast cancer), MDA-MB-231(breast cancer), Nalm-6 (acute lymphoblastic leukemia), L428 (Hodgkin lymphoma), Raji (Burkitt's lymphoma), Panc-1 (pancreatic cancer). These cell lines were maintained in DMEM or RPMI16, supplemented with 5-10% FBS and 1% penicillin/streptomycin in a humidified atmosphere with 5% CO2 at 37° C.

Construction of GFP-encoding plasmid and establishment of stable cell line: The LV-GFP-Puro plasmid was constructed and used to generate lentiviruses that carry the GFP reporter gene. Briefly, 293T packaging cells were seeded in 10 cm tissue culture plates. On the next day, the different plasmids (GFP-puro, VSVG, Pol-gag) were co-transfected, cells were incubated overnight, and cell media was replaced. 72-96 hours later, viruses were harvested and filtered using 0.45 μm pore filter. The viruses were supplemented with 8 μg/ml polybrene and used to infect HT29 cells. Following selection with puromycin, resistant colonies were identified.

Patient-derived tumor sample processing: Tumor biopsies were obtained, and primary tumor cells were isolated. Briefly, the representative material was harvested at the time of intervention. Small sections of maximally 1 mm\hat{ }3 were obtained using a sterile scalpel and all calcified parts were removed. The tumor tissue was digested using collagenase 2 (500 U/ml RPMI 1640) and DNase solution (22 KU/ml RPMI 1640). The remaining suspension was filtered through a 150 μm cell strainer. After centrifugation, the pellet was resuspended with erythrocyte lysis buffer (ELB) and culture medium was added to stop the reaction. After resuspension, the number of live cells/ml was counted by means of an automatic cell counter.

Cells were transplanted into the CAM model as described hereinabove and the resultant three-dimensional, vascularized organoid formed from primary culture was evaluated.

Growth of tumor cells using different matrices: Different types of matrices were examined for tumor growth as follows: 1) 0.8% Hyaluronic acid (Sigma); 2) E-C-L Cell Attachment Matrix (entactin-collagen IV-laminin) (Mercury); 3) Matrigel [extracted from the Engelbreth-Holm-Swarm mouse sarcoma, containing laminin (a major component, i.e. 56%), collagen IV, heparin sulfate proteoglycans, entactin/nidogen, and growth factors (EGF, bFGF, NGF, PDGF, IGF-1, TGF-1)].

Hematoxylin and Eosin (H&E) Staining and Immunohistochemistry (IHC) Staining Assay: Tumors were removed (using scissors and tweezers) from the CAM and transferred into 4% formaldehyde (FA). The excised tumors were frozen or fixed in 4% paraformaldehyde overnight at 4° C., and then placed into embedding cassettes followed by transfer into a tissue embedding station with an increasing graded alcohol series (50%, 70%, 80%, 95% ethanol, xylol and paraffin). Sections of the paraffin embedded tissues (3 μm) were deparaffinized by a decreasing graded alcohol series to double-distilled water (xylol, 95%, 80%, 70%, 50% ethanol, double-distilled water) and then used for histopathological analyses with hematoxylin and eosin (H&E) according to standard protocols.

Monitoring of CAM tumors growth via In Vivo Imaging System (IVIS) device: The use of fluorescently labeled cells allows monitoring tumor growth and response to therapy over time. The IVIS was used for monitoring the labeled tumors that were developed on the CAM. The eggs were placed in the device and live imaging was carried out.

### Example 1: Establishment of the Chorioallantoic Membrane (CAM) Model for Cell Lines

As illustrated in FIG. 2, by pulling 2 ml of egg white (albumen) from the bottom part of the egg, a successful separation of the CAM membrane from the eggshell was possible. This generated space at the top part of the egg for introducing the cancer cells. Importantly, this step was carried out on day 3 of embryo development as the attachment of the CAM to the shell inner membrane occurs around day 4-5. Thus, opening fertilized eggs for cultivation after day 3 will cause rupture of the shell associated with rupture of the CAM.

On day 7, the CAM was activated (at the site of transplantation) in order to increase the propensity of the membrane to generate new vascularization to the site of activation and cancer cells in suspension were transplanted at the top part of the CAM. The eggs were resealed and returned to the incubator. On day 9-10, 3D cancerous tumors were evident surrounded by a large vascularization network.

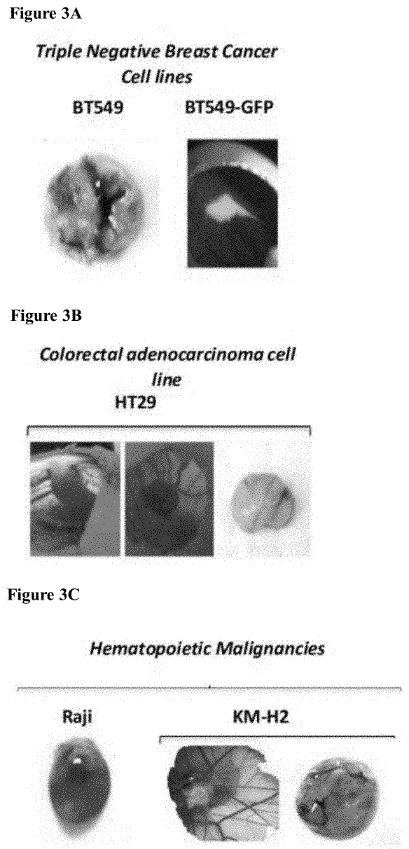

The efficiency and reproducibility of human cancer cell engraftment was demonstrated. Various cancer cell lines (indicated in Table 1, below), including hematopoietic malignancies and solid tumors (FIG. 3A-C) were successfully engrafted.

TABLE 1
cancer cell lines; N/S = data not shown

| Primary tissue | Cell line | Type of Cancer | Images |
|---|---|---|---|
| Colon | HT29 | Colorectal | FIG. 3B |
| | | adenocarcinoma | |
| Ascending | HCT116 | Colon carcinoma | N/S |
| colon | | | |
| Lung | A549 | Lung carcinoma | N/S |
| Lung | 1975 | Lung carcinoma, | N/S |
| | | NSCLC | |
| Prostate | DU145 | Metastatic prostate | N/S |
| | | cancer | |
| Prostate | PC3 | Metastatic prostate | N/S |
| | | cancer | |
| Breast | BT549 | Ductal Carcinoma | FIG. 3A |
| Breast | MDA- | Triple negative | N/S |
| | MB-468 | breast cancer | |
| Breast | MDA- | Metastatic breast | N/S |
| | MB-231 | cancer | |
| Breast | BT474 | Ductal carcinoma | N/S |
| Hematological | Nalm-6 | Acute lymphoblastic | N/S |
| malignancy | | leukemia (ALL) | |
| Hematological | L428 | Hodgkin lymphoma | N/S |
| malignancy | | | |
| Hematological | KM-H2 | Hodgkin lymphoma | FIG. 3C |
| malignancy | | | |
| Hematological | Raji | Burkitt's lymphoma | FIG. 3C |
| malignancy | | | |
| Pancreas | Panc-1 | Pancreatic cancer | N/S |

### Example 2: Comparison of Matrices as Grafting Material

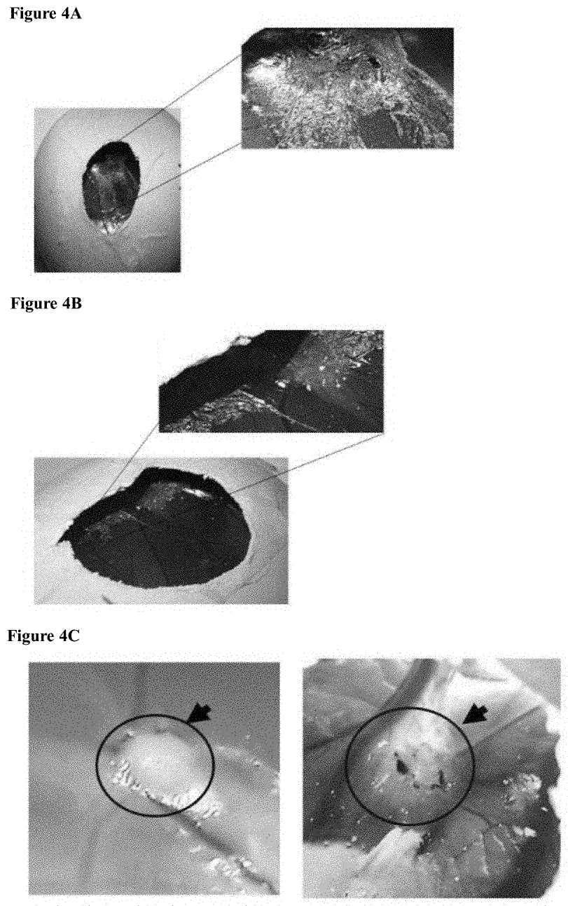

Although the basic CAM model allows for take (engraftment) and growth of human cancer cells, in order to yield optimal results different extracts of basement membrane were tested. In specific, Matrigel, Hyaluronic acid (HA) and E-C-L (entactin-collagen IV-laminin) Cell Attachment Matrix were tested. HT29 cancer cells were implanted as described hereinabove with mixed (1:1 by volume) with HA, ECL matrix or Matrigel. Both HA alone (FIG. 4A) and ECL matrix (FIG. 4B) did not produce detectable tumors in the CAM model. In contrast, Matrigel was found to enhance the tumorigenicity of human cell lines, producing readily detectable tumors from all cell lines tested (FIG. 4C, Table 1). Notably, when tumor specimens were engrafted with 0.8% HA supplemented with bFGF and VEGF this combination was also found to be effective. Subsequently, various other cancer types were also tested, and these results were found to be consistent across various cancers. As such, all future engraftments were supplemented with these two growth factors.

### Example 3: Characterization of CAM-Based Xenograft

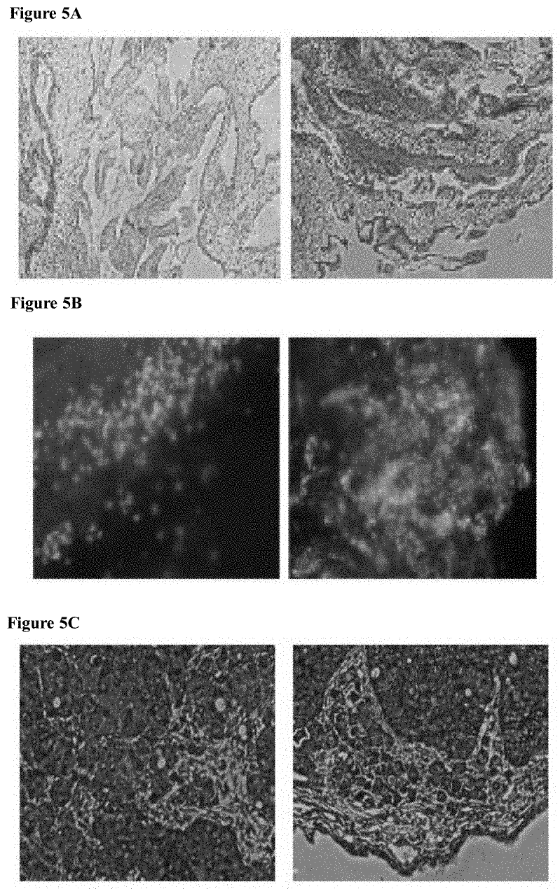

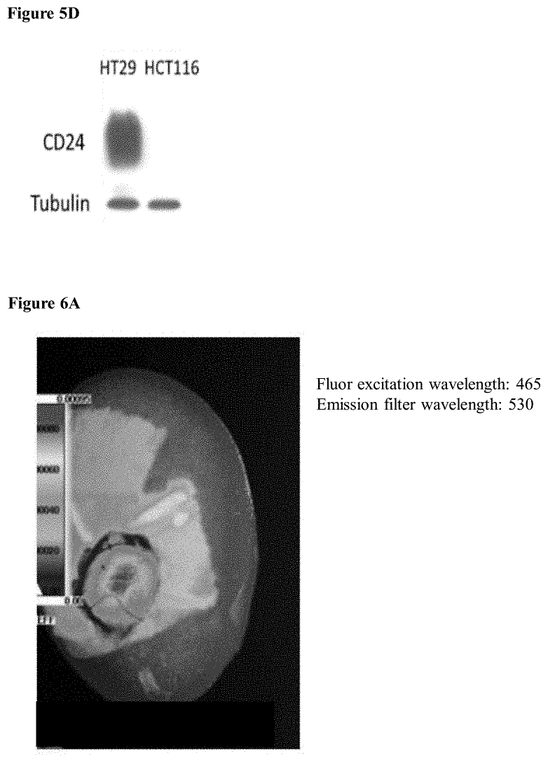

To confirm engraftment a GFP positive colorectal cancer cell line HT29-GFP was implanted in the CAM model. Using fluorescently tagged cells allows for easy cell tracking. FIGS. 5A and 5B illustrate histological sections and frozen sections of HT29-GFP derived xenograft, respectively. HT29 is known to express the GPI-anchored mucin like protein CD24. Immunohistochemistry (IHC) using the anti-CD24 mAb demonstrated that the cancer cells continued to express this important marker while growing in the CAM model (FIG. 5C). After excision of the tumor CD24 levels as a marker for cell number/tumorigenicity was easily quantifiable (FIG. 5D).

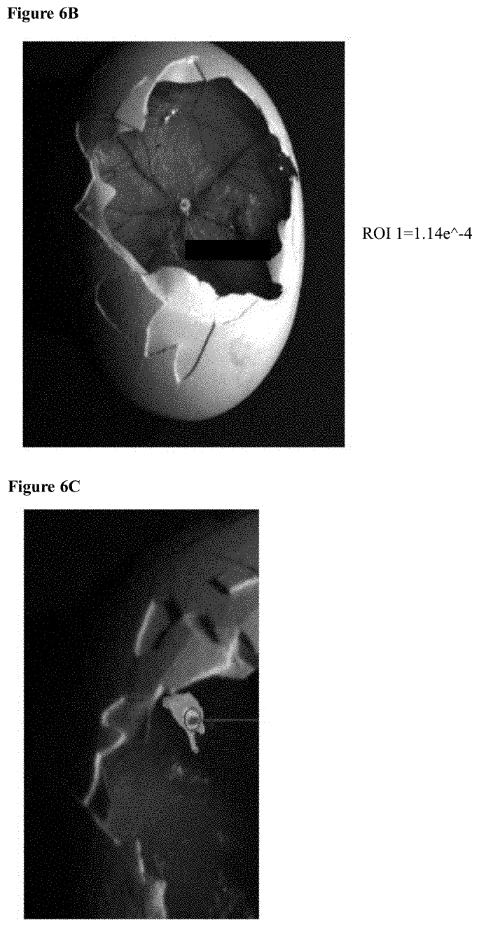

In addition to using conventional markers for characterizing the tumor after excision, the CAM based model allows measurement of the tumor in real time as it grows/responds to treatment. This was done using an In Vivo Imaging System (IVIS), a known fluorescent imaging platform. Regions of interest were defined using an automatic intensity contour procedure to identify fluorescence signals with intensities significantly greater than the background (FIG. 6A-C). In-ovo imaging of GFP intensity was possible even at very early stages of tumor development (FIG. 6B-C).

### Example 4: Establishment of the CAM Model for Primary Tumor Cells for Use in Personalized Cancer Therapy

Immortalized cancer cell lines cannot meet the needs of personalized medicine. Immortal cancer cell lines poorly represent the diversity, heterogeneity and drug-resistant tumors occurring in patients. Therefore, culture of primary cells from solid and hematologic malignancies has thus gained significant importance in personalized cancer therapy.

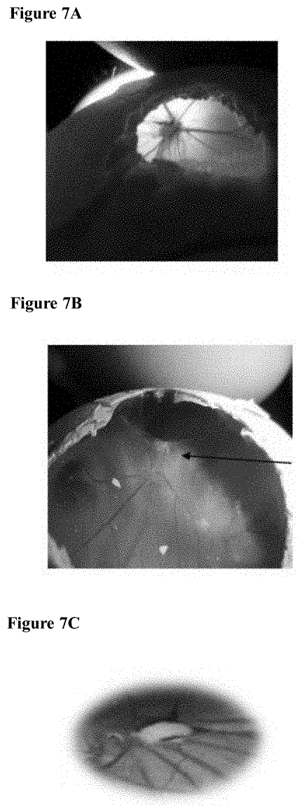

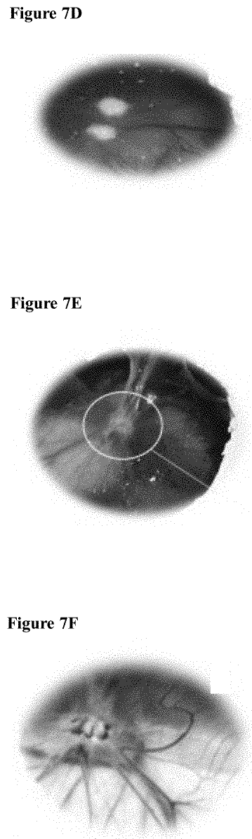

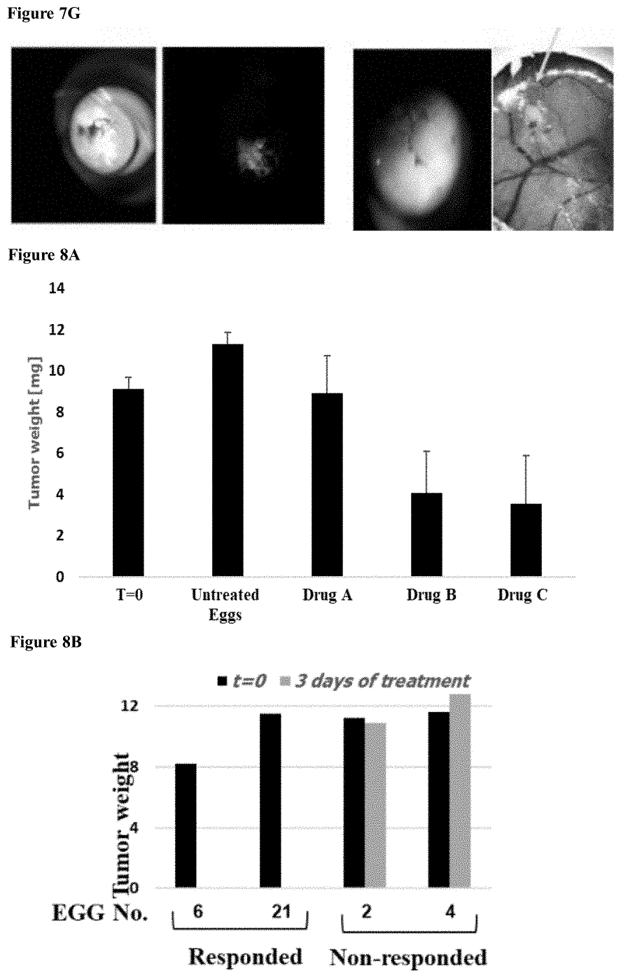

To test whether the CAM model can be used for culturing and organoid generation from primary cancer samples, single cell suspensions of primary cancer cells were prepared (see Materials and Methods). The cells were mixed 1:1 by volume with Matrigel and supplemented with growth factors. The CAM was activated as before and the cells were placed on the activated CAM. Primary cells from CLL (FIG. 7A), colon cancer (FIG. 7B), ovarian cancer (FIG. 7C), uterine cancer (FIG. 7D), breast cancer (FIG. 7E) and vulval cancer (FIG. 7F) were all tested and found able to engraft and to form organoids.

Importantly, it was also found that the CAM model can be used for repetitive culturing of the CAM-grown tumors (FIG. 7G). HT29-GFP stable cells were transplanted onto the CAM as before. 7 days later, the established tumors were removed and re transplanted onto the CAM of a new egg. This experiment was also performed with primary colorectal cancer samples from patients and similar results were observed. The initial graft can thus be successfully serially passaged for multiple generations, allowing for the maintenance of primary cancer samples in perpetuity.

### Example 5: Evaluation of Drug Delivery Route

There are several inoculation sites for therapeutic delivery in embryonated chicken eggs: injection into the chorioallantois, into the embryo, onto the CAM, into the amnion or into the yolk sac. It was surprisingly found that intravenous (IV) delivery can be mimicked by injection into the yolk sack. This enables a simple, efficient and fast route of administration that allows the therapeutics to be delivered to the cancer tissue through the vasculature, without affecting the survival of the chick embryos. Chemotherapeutic drugs were tested as single agents via administration to the yolk sac. Of the three tested drugs, two were found to inhibit tumor development of colorectal cancer cell line cells (FIG. 8A) and tumor specimens (data not shown).

It was also found that the CAM model can be used to evaluate tumor heterogeneity. A single tumor was dissociated into a single cell suspension and cells were transferred to a large number of eggs. When chemotherapeutics were tested, cells in certain eggs, but not in others were found to respond (FIG. 8B) demonstrating the usefulness of the system in evaluating heterogeneity and potentially selecting combination therapies.

### Example 6: Establishment of “Humanized Egg” Resembling the Mouse PDX Model

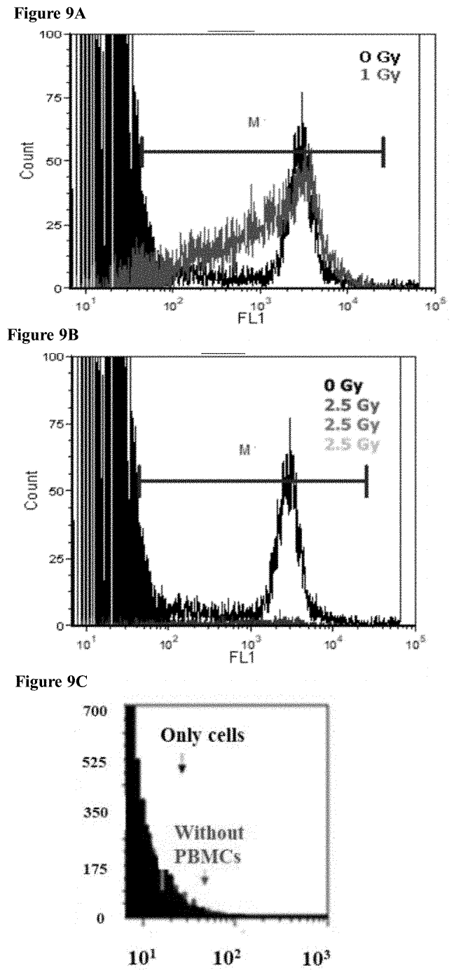

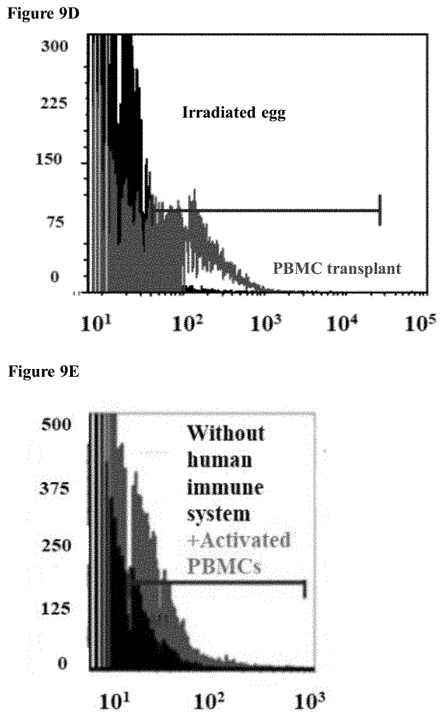

As chemotherapeutics do not require the presence of an intact immune system to kill cancer cells, they could be tested in this CAM model. However, in order to test immunotherapeutics a human immune system is required. To this end a “humanized egg” was created. Although the developing egg is initially immunodeficient, the chicken immune system does begin to form and by day 15 is at least partially functional. In a humanized CAM model, it would be essential 1) that the effect of the human immune cells is measured without contamination by chicken immune cells and 2) that the chicken immune cells not try and reject the human immune cell transplant. As such, it was necessary to first completely ablate the avian immune system. This was done by irradiating the egg on day 5 or 6 (EDD=5-6, which was theorized to completely kill all progenitor cells of the immune system. At 1 Gray (1.0 Gy, rate of 15/sec) of radiation the immune system was not substantially affected and CD45+ cells were still highly present (FIG. 9A). When 2.5 Gy was used (2.5 Gy, rate of 15/sec), no chicken CD45+ cells were detected in irradiated eggs as compared to naïve eggs at about EDD 18 (FIG. 9B). Next, the irradiated egg was administered patient-derived immune cells (e.g., PBMCs) at EDD 8-9. The presence of human immune cells was confirmed in blood taken from the chicken egg, based on FACS detection of human CD45+ cells (FIG. 9C-D). Specifically, cells of the human immune system were detectable in the chicken egg as early as 2 days after immune-reconstitution, these cells were shown to be not just CD45+, but also CD8+(FIG. 9E).

Importantly, the irradiated egg was still transplantable with the various cancer types which had been explanted in the non-irradiated egg (see Example 1). Indeed, the loss of the forming immune system may have enhanced take of the cancer cells as in the irradiated egg model successful transplantation reached a rate of ˜90% which is superior to what is known in the literature.

### Example 7: Immunotherapy Testing in the Humanized Egg

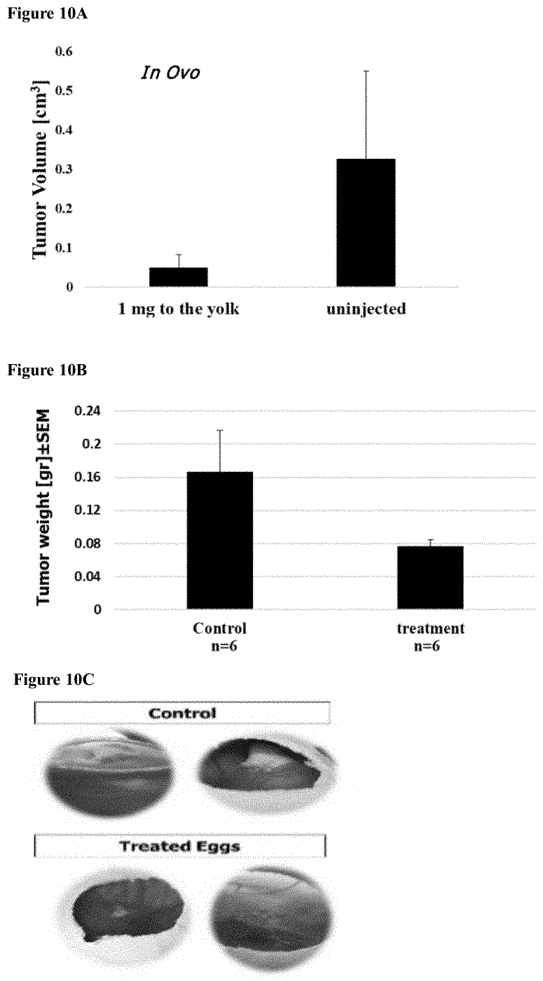

Antibody immunotherapy could now be tested as the model comprises human immune cells. Anti-CD24 antibody was IV administered by injection into the yolk sac and the effect on tumor weight was monitored. At the dose tested the antibody in conjunction with the immune cells produced a robust anti-tumor effect by day 3-4 after administration of the antibody (FIG. 10A). The effect of the antibody was similar to that reported in humanized mice.

Next, cell therapy in the form of CAR-T cells was tested. Breast cancer cells were transplanted to the CAM after irradiation. CAR-T cells were generated with receptor specificity to a breast cancer antigen and added in close proximity to the cancer cells. The CAR-T cells produced a significant decrease in tumor weight after 3 days (FIG. 10B) across all the tested organoids (n=6) (FIG. 10C).

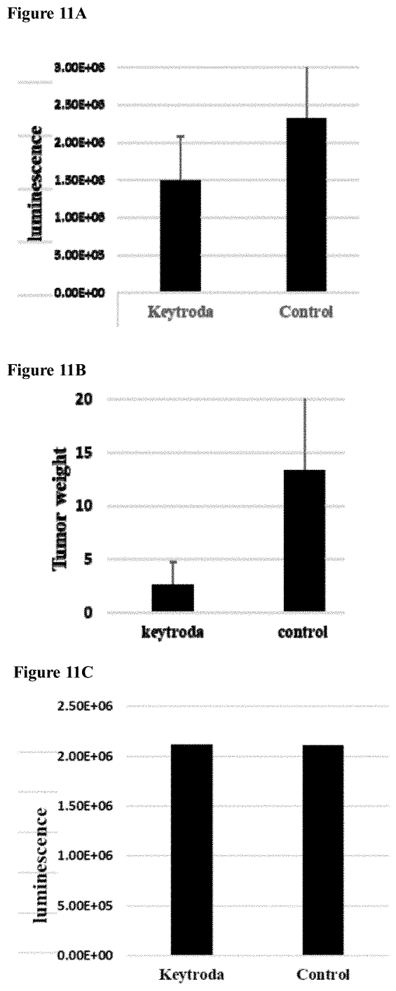

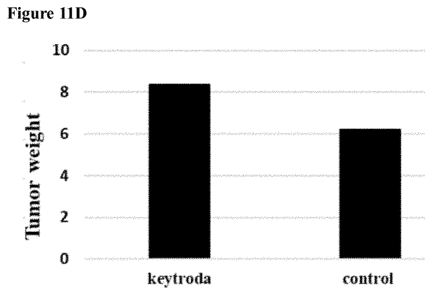

Finally, the combination of immunotherapy and cell therapy was successfully tested. Keytruda, an anti-PD-1 immune checkpoint inhibitor, was IV administered (by administration to the yolk sac) while CAR-T cells were applied topically onto the CAM, in close proximity to the tumor (as discussed hereinabove). Keytruda is known to be effective against microsatellite instability (MSI)-high colorectal tumors, but ineffective against MSI-low tumors. Primary cells of both tumor types from patients were transplanted (EDD=7) into the irradiated CAM model (irradiation at EDD=5). CAR-T cells were added (EDD=9) followed by Keytruda administration (EDD=10). The size of the tumors was monitored in vivo by luminescence and the tumors were weighed after excision. In order to allow time for the therapeutic effect to be observable and significant, it was necessary to extend growth of the CAM model well beyond when the chicken immune system would normally begin to be functional. However, as the eggs were irradiated the chicken immune system could not form and the therapeutic effect could be accurately measured at much later time points. As illustrated in FIGS. 11A-D, colorectal MSI-high tumors (CRC-MSI-High) responded to Keytruda/CAR-T treatment and showed a significant decrease in tumor size in vivo (FIG. 11A) and when weighed after excision (FIG. 11B). In contrast, colorectal MSI-low tumors (CRC-MSI-Low) did not respond to treatment (FIG. 11C-D). These results demonstrate the usefulness of the CAM model in studying various forms of immunotherapy, including combination therapies. Further, they point to the essential need for ablation of the chick immune system. Besides the potential for the burgeoning immune system to interfere with data collection, the use of transplanted immune cells in combination with cancer therapeutics (immunotherapies) pushes the window for data collection well beyond when the chicken immune system is fully functional. To properly model the effects of immunotherapies on personal tumor samples, the old CAM model is not sufficient, and the irradiated model is greatly preferred.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A xenograft egg model comprising:
a. a viable fertilized non-mammalian egg, wherein said egg comprises an ablated immune system;
b. a first plurality of mammalian cells in contact with a vasculature of said egg; and
c. a second plurality of mammalian cells in contact with a vasculature of said egg, wherein said second plurality of cells comprises immune cells from the same species as said mammalian cells of said first plurality.

2. The xenograft egg model of claim 1, wherein said non-mammalian egg is:
a. an avian egg,
b. a reptilian egg; or
c. chicken egg.

3. (canceled)

4. The xenograft egg model of claim 1, wherein at least one of:
a. said first plurality of mammalian cells are in contact with a chorioallantoic membrane (CAM) of the egg, said second plurality of mammalian cells are in contact with a CAM of the egg or both;
b. said first plurality of mammalian cells and said second plurality of mammalian cells are derived from the same subject;
c. said first plurality of mammalian cells and said second plurality of mammalian cells are not in direct contact;
d. said first plurality of mammalian cells, said second plurality of mammalian cells, or both are comprised in a composition which comprises an exogenous matrix material, exogenous angiogenic growth factors or both, optionally wherein said matrix material comprises hyaluronic acid (HA) or is Matrigel or wherein said growth factors comprise bFGF and VEGF;
e. said first plurality of mammalian cells comprises diseased cells;
f. said first plurality of mammalian cells comprise cancer cells; and
said first plurality of mammalian cells comprise colon cancer, colorectal cancer, lung cancer, prostate cancer, breast cancer, pancreatic cancer, liver cancer, kidney cancer, skin cancer, thyroid cancer, throat cancer, head or neck cancer, brain cancer, ovarian cancer, cervix cancer, spleen cancer, lymphoid cancer or hematopoietic cancer cells.

5. (canceled)

6. (canceled)

7. (canceled)

8. (canceled)

9. (canceled)

10. (canceled)

11. (canceled)

12. The xenograft egg model of claim 1, wherein said immune cells comprise peripheral blood mononuclear cells (PBMCs).

13. (canceled)

14. The xenograft egg model of claim 1, wherein said egg comprising an ablated immune system is substantially devoid of CD45+ non-mammalian cells.

15. The xenograft egg model of claim 14, wherein said egg is a chicken egg and remains devoid of CD45+ non-mammalian cells beyond day 15 of development.

16. The xenograft egg model of claim 1, wherein said egg comprising an ablated immune system is an irradiated egg.

17. (canceled)

18. A method of generating a xenograft egg model, the method comprising:
a. providing a viable fertilized non-mammalian egg;
b. irradiating said fertilized egg;
c. placing a first plurality of mammalian cells in contact with vasculature of said fertilized egg; and
d. placing a second plurality of mammalian cells in contact with vasculature of said fertilized egg wherein said second plurality of mammalian cells comprises immune cells from the same species as said mammalian cells of said first plurality,
thereby producing a xenograft egg model.

19. The method of claim 18, wherein said egg comprises an eggshell and further comprising prior to (b) removing at least a part of egg albumen from a bottom region of said fertilized egg so as to separate a chorioallantoic membrane (CAM) of said fertilized egg from said eggshell; and creating an aperture at a top region of said eggshell to expose at least a portion of said CAM of said fertilized egg, optionally wherein said removing, said creating an aperture or both is performed on embryo development day 1-3.

20. (canceled)

21. The method of claim 18, wherein at least one of:
a. said first plurality of mammalian cells, said second plurality of mammalian cells or both are placed in contact with said CAM;
b. said first plurality of mammalian cells and said second plurality of mammalian cells are derived from the same subject;
c. said first plurality of mammalian cells and said second plurality of mammalian cells are not in direct contact;
d. said first plurality of mammalian cells, said second plurality of mammalian cells, or both are comprised in a composition which comprises an exogenous matrix material, exogenous angiogenic growth factors or both, optionally wherein said matrix material comprises hyaluronic acid (HA) or is Matrigel or wherein said growth factors comprise bFGF and VEGF;
e. said first plurality of mammalian cells comprises diseased cells;
f. said first plurality of mammalian cells comprise cancer cells; and
g. said first plurality of mammalian cells comprise colon cancer, colorectal cancer, lung cancer, prostate cancer, breast cancer, pancreatic cancer, liver cancer, kidney cancer, skin cancer, thyroid cancer, throat cancer, head or neck cancer, brain cancer, ovarian cancer, cervix cancer, spleen cancer, lymphoid cancer or hematopoietic cancer cells.

22. The method of claim 18, wherein said irradiating is at least one of:
a. performed on embryo development day 4 to 7;
b. comprising irradiating at 1.5-3.5 Gy at a rate of about 10-20 rad/sec; and
c. comprising irradiating at 2.5 Gy at a rate of about 15 rad/sec.

23. (canceled)

24. (canceled)

25. The method of claim 21, further comprising activating said CAM prior to placing said first plurality of mammalian cells, said second plurality of mammalian cells or both in contact with said CAM.

26. The method of claim 18, wherein at least one of:
a. said placing said first plurality of mammalian cells is performed on embryo development day 6 to 8;
b. said placing a second plurality of mammalian cells is performed on embryo development day 8 to 10.

27. (canceled)

28. (canceled)

29. (canceled)

30. (canceled)

31. (canceled)

32. (canceled)

33. (canceled)

34. (canceled)

35. The method of claim 18, wherein said immune cells comprise peripheral blood mononuclear cells (PBMCs).

36. (canceled)

37. A xenograft egg model produced by a method of claim 18.

38. A method of screening a therapeutic agent, the method comprising:
a. providing the xenograft egg model of claim 1;
b. contacting said first plurality of mammalian cells with said therapeutic agent;
c. analyzing at least one response parameter in said first plurality of mammalian cell; and
d. selecting a therapeutic agent that produces a desired change in said at least one response parameter;
thereby screening a therapeutic agent.

39. The method of claim 38, wherein said mammalian cells of said first plurality comprise diseased cells, said at least one response parameter comprises a disease phenotype or number of disease cells and said desired change is a reduction in disease phenotype or number of disease cells.

40. The method of claim 38, wherein said therapeutic is an immunotherapeutic, optionally selected from an immune checkpoint inhibitor, an antibody against a cancer surface antigen and a modified immune cell, optionally wherein said modified immune cell is a CAR immune cells and said CAR immune cell is a part of said second plurality of mammalian cells.

41. (canceled)

42. (canceled)

43. The method of claim 38, wherein said contacting said first plurality of mammalian cells with said therapeutic agent is performed on embryo development day 9 or 10, said analyzing is performed on embryo development day 15 or beyond, or both.

44. The method of claim 38, wherein said therapeutic agent is injected into a yolk sac of said fertilized egg.

45. (canceled)
